(19) United States
(12) Patent Application Publication
(10) Pub. No.: US 2012/0029367 A1
(43) Pub. Date: Feb. 2, 2012

(54) HEART RATE WATERPROOF MEASURING APPARATUS

(76) Inventor: Hind Louis HOBEIKA, Yarze (LB)

(21) Appl. No.: 13/167,044

(22) Filed: Jun. 23, 2011

(30) Foreign Application Priority Data
Jul. 31, 2010 (LB) 9099

(51) Int. Cl. A61B 5/02

(52) U.S. Cl. 600/500

(57) ABSTRACT

A biofeedback device and the reflected infrared sensor used thereby are described herein that can be mounted on or integrated with eyewear such as swimming goggles. The biofeedback device includes a heart rate measuring apparatus comprising a reflected infrared sensor, a microcontroller comprising one or more filters and one or more amplifiers, a power source in electrical communication with the heart rate measuring apparatus, and a user interface. The reflected infrared sensor is positionable to detect heart rate from the temporal artery in the head. Heart rate is then communicated to the user by one or more tactile, auditory, or visual signal elements, such as a light-emitting diode display mounted within the goggles so as to be visible to the user while swimming.

### CROSS-REFERENCE TO RELATED APPLICATION

This application claims the benefit of the following foreign application, which is incorporated herein by reference in its entirety: Lebanese Serial Patent No. 9099, filed Jul. 31, 2010.

### STATEMENT REGARDING FEDERALLY SPONSORED RESEARCH OR DEVELOPMENT

n/a

### FIELD OF THE INVENTION

The present invention relates to a waterproof heart rate measuring apparatus that can be mounted on or integrated with eyewear such as swimming goggles.

### BACKGROUND OF THE INVENTION

Heart rate monitoring is one of the most important tools for efficient cardiovascular training. As an indicator of not only the level of physical exertion but also the body's physiological adaptation to exercise, heart rate is a basis on which to gauge overall fitness. Additionally, monitoring heart rate is an easy way to make sure the body is not being dangerously overexerted. Many types of heart rate monitoring devices are known in the art, including devices that are worn around the wrist, on a finger, or around the torso, and those that use pressure, light, electrodes, and other methods to measure heart rate.

Heart rate is defined as the number of heart beats per unit of time, usually expressed as beats per minute (bpm), and can change as the body's need for oxygen changes in response to activity. The maximum heart rate, defined as the maximum safe heart rate for an individual, depends on factors such as age, sex, and fitness level of the individual. The most accurate way of measuring the maximum heart rate is through a cardiac stress test, in which the individual exercises while being monitored by an electrocardiograph (EKG). For general purposes, however, a formula is used to estimate Maximum Heart Rate:

HRₘₐₓ=220−age.

There is a direct relationship between heart rate and intensity of physical activity. Three different training zones are commonly used: weight loss, fitness, and maximum performance. If an individual wishes to lose weight, the individual should limit heart rate to 50% to 70% of the individual's maximum heart rate during exercise. To increase fitness, an individual should limit heart rate to 70% to 85% of maximum heart rate. An individual who wants to improve athletic performance should aim for a heart rate that is higher than 85% of the individual's maximum heart rate. In professional athletic training, an athlete may utilize all three heart rate zones for building cardiovascular health and endurance.

A number of heart rate sensors are known, including those that use sound, light, and/or pressure to measure the pulse. One type of sensor is an infrared plethysmograph. Such a sensor includes a photodiode that emits an infrared light and a phototransistor that receives the reflected infrared light. The superficial temporal artery, a major artery of the head that is located approximately 5 mm below the skin of the temple, is commonly used for heart rate measurement. It is the smaller of the two branches of the external carotid artery, and its pulse is palpable superior to the zygomatic arch and anterior to and superior to the tragus. The pulse is calculated from the changes in volume of the temporal artery between the systole and diastole phases. In the diastole phase, the cavities of the heart are expanded and fill with blood, resulting in low arterial blood pressure. The heart contracts in the systole phase, resulting in higher blood pressure. The amount of blood in an artery is directly related to its volume: more blood (higher volume) in the systole phase and less blood (lower volume) in the diastole phase. There is a slight increase in the infrared light absorption by the artery during the systolic phase, and less light is reflected back to the phototransistor of the sensor.

Athletes and participants in every sport can benefit from monitoring heart rate during training, including swimmers. Taking accurate and frequent heart rate measurements not only is useful in tracking changes in cardiovascular fitness over time and optimizing training, but also to prevent injury and exercise stress. If not correctly monitored, a swimmer can easily overtrain, which means that heart rate is so high that the swimmer is training in an anaerobic zone. Although anaerobic training can be a part of a balanced training program, an anaerobic workout can damage the muscle cell walls and result in decreased aerobic capacity for 24 to 96 hours. Consistently training in the anaerobic zone is counterproductive and can lead to injury and fatigue. The traditional method of measuring heart rate is to count the number of pulses over one minute. Heart rate measurements are of the greatest training value when measured during the physical activity, but it is difficult to accurately measure swimming heart rate using the wrist or neck pulse because of human error and the inconvenience of having to stop swimming long enough to measure heart rate. A heart rate monitoring device is preferable, but the device options are limited by the additional need for waterproofing and a practical means of communicating heart rate and other biofeedback data.

An effective heart rate monitor for swimmers must also be able to communicate current heart rate to the user in a way that does not disrupt training. Devices worn on the wrist, for example, are inconvenient because the user cannot see the display while swimming. Other devices may be able to display a number in the user's field of view, but the user must still concentrate enough to read the numbers. This may not be an easy task while the user is swimming quickly or is focused on stroke technique.

Also, some swimmers use certain training devices that do not interrupt swimming, such as pacing devices, timers, and lap counters. However, no device offers a combination of a heart rate monitor, pacing device, timer, lap counter, and other features such as pulse oximetry and calorie monitoring. Furthermore, no device displays heart rate to the user in a non-numeric method that the user can interpret easily while swimming.

It would therefore be advantageous to provide a waterproof heart rate monitoring device that is convenient to use during swimming and also is capable of measuring and recording other types of biofeedback and non-biofeedback data. For example, the microcontroller 34 of the device may additionally comprise circuitry for performing the functions of a chronometer, timer, lap counter, distance measurement device, calorie counter, blood oximeter, and wireless transmitter (such as a Bluetooth® device). It would also be desirable that the device should include a method of wireless transmission so the measured biofeedback and non-biofeedback data could be sent from the device to a mobile phone or computer, or include an integrated memory chip that stores the data. Further, such a device should communicate heart rate to the user without requiring the user to divert attention away from training.

### SUMMARY OF THE INVENTION

The present invention advantageously provides a biofeedback device, and the reflected infrared sensor used thereby, that can be mounted on or integrated with eyewear such as swimming goggles. The biofeedback device may comprise a heart rate measuring apparatus may measure the user's heart rate using a reflected-infrared plethysmograph (reflected infrared sensor) that detects heart rate from the temporal artery in the head. The reflected infrared sensor may transmit the detected heart rate signal to one or more amplifiers, one or more filters, and a microcontroller, which calculates the final heart rate measurement.

The heart rate measuring apparatus may also include a user interface by which the user can enter age, weight, target heart rate value or zone, and other data, or the heart rate measuring apparatus may be connected to a wireless interface (such as WiFi, infrared, or Bluetooth®) to incorporate a wireless user interface housed in a remote device. After the heart rate is measured, the measurement may be processed by a comparator that compares the user input values and the heart rate measurement. The heart rate measuring apparatus also may include circuitry that allows it to measure and record other biofeedback and non-biofeedback data such as calories burned and blood oxygen, and also data such as time, swim pace, swim duration, distance traveled, and laps completed.

The result of this comparison is communicated to the user by one or more signal elements, such as a display of colored light-emitting diodes (LEDs) on the inside of the goggles, the one or more signal elements notifying the user whether he should accelerate, decelerate, or maintain the current pace. The signal notification scheme may consist of LEDs of three or more colors, such as one color for each training zone (for example, weight loss, fitness, and maximum performance), with a blinking red color displayed when no heart rate is detected. Additionally, the lights may glow steadily or may blink at a variable rate depending on whether the user should speed up, slow down, or maintain the current pace to keep the user's heart rate within the desired training zone.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a perspective view of a first embodiment of the heart rate waterproof measuring device;

FIG. 2A shows a perspective view of a waterproof housing with the reflected infrared sensor contained therein;

FIG. 2B shows a sectional view of the reflected infrared sensor within the housing, the reflected infrared sensor being covered by a thin waterproof layer of material;

FIG. 3 shows a second embodiment of the heart rate waterproof measuring device;

FIG. 4 shows an alternate sectional view of the device of FIG. 3;

FIG. 5 shows a cross-sectional view of the reflected infrared sensor of the device and placement of the reflected infrared sensor on the skin above the temporal artery of the head;

FIG. 6A shows a cross-sectional view of the waterproof housing including an reflected infrared sensor and panel-type sensor adjustment mechanism;

FIG. 6B shows a sectional elevation view of the waterproof housing including the reflected infrared sensor and panel-type sensor adjustment mechanism;

FIG. 6C shows the spiral-type sensor adjustment mechanism;

FIG. 7A shows a sectional view of the device having rope-type LEDs located on the circumference of the inner surface of a lens;

FIG. 7B shows a sectional view of the device having discrete LEDs located on the inner surface of a lens;

FIG. 8A shows a sectional view of the device having a signal element coupled to a eye cup track positionable element;

FIG. 8B shows a sectional view of the device having a signal element coupled to a suction cup positionable element; and

FIG. 9 shows a schematic diagram of an exemplary function of the device.

### DETAILED DESCRIPTION OF THE INVENTION

Monitoring heart rate is very important in an athletic training program, especially swimming. Although there are many available types of heart rate monitors, not all are waterproof and convenient for use while swimming. Furthermore, none of the available waterproof heart rate monitors combine a heart rate measuring apparatus with the measurement of time, calories burned, swim pace, swim duration, blood oxygen, distance traveled, and laps completed. The present invention advantageously provides a biofeedback device that can be waterproofed and mounted on or integrated with eyewear such as swimming goggles. Heart rate is then communicated to the user by one or more signal elements positioned within the user's field of vision (if visual), or otherwise communicated to the user (if auditory or tactile). The present invention also advantageously provides a reflected infrared sensor used within the device, the reflected infrared sensor having optimal geometry for detecting heart rate from subcutaneous blood vessels, such as the superficial temporal artery.

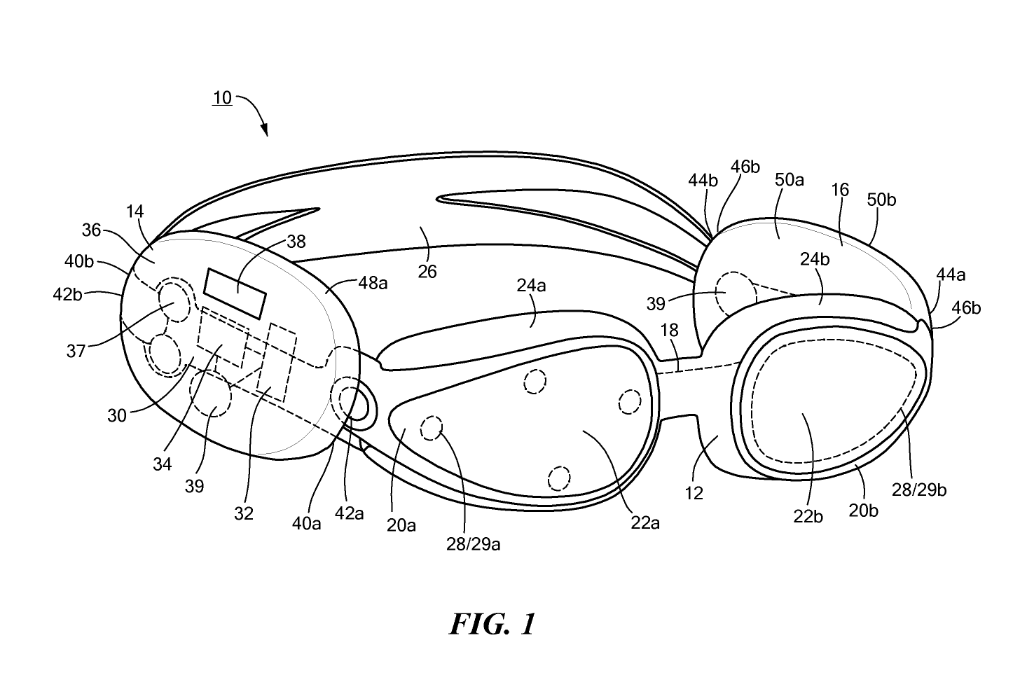

Referring now to FIG. 1, a first embodiment of the biofeedback device 10 is shown. The biofeedback device 10 may comprise a pair of goggles 12, a first waterproof housing 14, a second waterproof housing 16, and one or more wires 18 for electrical communication between the first and second waterproof housing 14, 16. The goggles 12 may be a pair of traditional swimming goggles, or they may be any other type of protective eyewear. The goggles 12 may comprise a first and second eye cup 20a, 20b, a first and second lens 22a, 22b, a first and second eye cup gasket 24a, 24b, and a head strap 26. The first and second eye cups 20a, 20b may be composed of any transparent or semi-transparent material, including polycarbonate, optical-grade plastic, or even glass. The first and second eye cup gaskets 24a, 24b may be composed of any material suitable for contact with the face, although silicone and foam are the most popular materials. However, the goggles 12 may not include the first and second eye cup gaskets 24a, 24b, as seen in Swedish goggles commonly used for competitive swimming. One or more signal elements 28, such as LEDs 29, either rope-type (29a) or discrete LEDs (29b), may be included within the interior of the eye cup. The one or more signal elements 28 may comprise any type of visual, auditory, or tactile signaling system that can communicate heart rate, pace, or other measurements to the user, and may communicate such in a non-alphanumeric manner.

The one or more signal elements 28 shown in the figures is an LED system, and the LEDs 29 are discussed in more detail below. The head strap 26 may also be of any suitable material, although the most popular materials are silicone and rubber (which are resilient) and the typical bungee cord (a cord with a core composed of a plurality of elastic strands, covered in a woven polypropylene or cotton sheath). The head strap 26 may comprise a single strap, a split single strap, a double strap, or any variation that will securely hold the goggles 12 to the user's head.

Continuing to refer to FIG. 1, the first waterproof housing 14 may contain therein the heart rate measuring apparatus 30 comprising a reflected infrared sensor 32, a microcontroller 34, and a user interface 36. Although the term “heart rate measuring apparatus 30” is used herein for simplicity, it should be understood that the heart rate measuring apparatus 30 also may include circuitry that allows it to measure and record, in addition to heart rate, other biofeedback and non-biofeedback data such as calories burned and blood oxygen, and also data such as time, swim pace, swim duration, distance traveled, and laps completed. The user interface 36 may comprise one or more buttons 37 and one or more display screens 38, or it may additionally or alternatively comprise any other operable elements such as knobs, switches, touch screens, etc. The microcontroller 34 of the heart rate measuring apparatus 30 calculates the heart rate. The reflected infrared sensor 32 transmits signals of voltage per unit of time to the microcontroller 34, which may comprise one or more filters that filter all noise coming from electromagnetic interference and from ambient or environmental light and one or more amplifiers that amplify the remaining signal. The microcontroller 34 may then digitally filter the signal to extract the alternating current (AC) component of the signal, and then evaluate the time (T) between two pulses. The microcontroller 34 follows a formula to calculate the heart rate:

Heart Rate=60/T

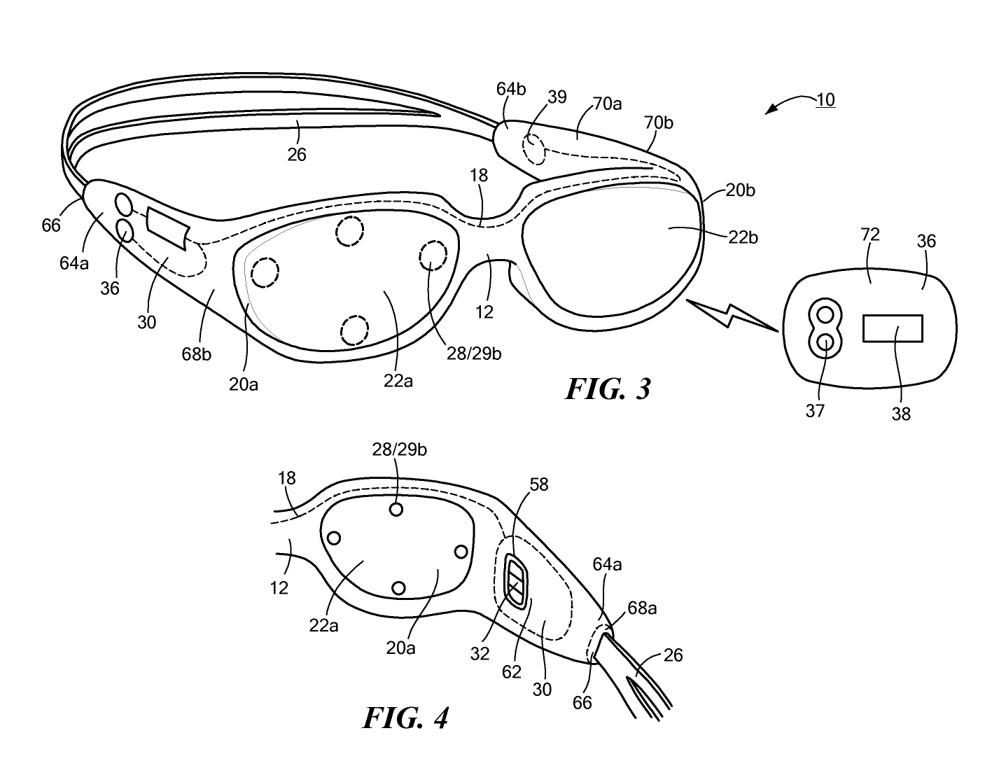

To obtain an accurate measurement over time, every five heart rate measurements may be averaged by the microcontroller 34 to obtain a moving average heart rate. A comparator may compare between the heart rate measurement and the target heart rate (calculated by the microcontroller 34 based on data entered in the user interface 36). Further, the microcontroller 34 may include a wireless communication interface adapted to be in wireless communication with a wireless data network, enabling transmission of recorded data to a computer, mobile phone, or other wireless device, or an integrated memory chip. The user interface 36 may also be in wireless communication with a wireless remote keyboard and display device, such as a dedicated device, mobile phone, PDA, or any other suitable device that is operable on wireless networks such as Bluetooth® or Wi-Fi. Additionally, the user interface 36 may be disposed within the first waterproof housing 14, or it may be housed in a remote device 72 in wireless communication with the microcontroller 34 (shown in FIG. 3). For simplicity, the term “microcontroller” as used herein may include the one or more filters, one or more amplifiers, comparator, wireless interface, and any other circuitry used to receive signals from the reflected infrared sensor 32 and perform calculations to produce final measurements and communicate said measurements to the user through a display element 28.

Continuing to refer to FIG. 1, the second waterproof housing 16 may contain therein a power source 39 that may be rechargeable or single use, for example a small battery such as a hearing aid or watch battery (button cell). The first and second waterproof housings 14, 16 may be composed of any rigid or semi-rigid, lightweight, waterproof material, such as acrylic, to prevent water and humidity from entering the housing and coming in contact with the electronic elements, to protect the unit against shock damage (such as when the biofeedback device is dropped), and to increase stability to ensure accurate heart rate measurements. The housing shape may be oval or rounded to increase hydrodynamic efficiency, and the first and second waterproof housings 14, 16 each may include a mechanism (such as with a latch or screws) by which the user may open the waterproof housing to change the power source 39, adjust the reflected infrared sensor 32, or make repairs. All measurements taken by the reflected infrared sensor 32 rely on the accurate emission, reflection, and reabsorption of infrared light. Therefore, it is important to exclude as much ambient or environmental light as possible. To achieve this, the housing may further be coated with a layer of opaque material to block any interference by ambient or environmental light.

One or more wires 18 may put the first and second waterproof housings 14, 16 in electrical communication with each other and with the one or more signal elements 28 (if wireless communication is not used). These wires 18 may be disposed within a chamber defined by the frame of the goggles 12 that extends between the first and second waterproof housings 14, 16 and the one or more signal elements 28. The wires 18 and may be rigid enough to be easily fed through the chamber so the waterproof housings 14, 16 and one or more signal elements 28 may be completely removed from the goggles 12. Furthermore, the wires 18 may be coupled to a connection means on both ends so the wires 18 can be readily connected and disconnected from the waterproof housings 14, 16 and one or more signal elements 28.

Continuing to refer to FIG. 1, the first and second waterproof housings 14, 16 may be held securely against the skin of the user by the head strap 26, and the user may position the first and second housings for comfort and accuracy. The first waterproof housing 14 may have a first end 40a including a first strap attachment means 42a and a second end 40b including a second strap attachment means 42b, and the second waterproof housing 16 may have a first end 44a including a first strap attachment means 46a and a second end 44b including a second strap attachment means 46b, each strap attachment means 46a, 46b defining an opening through which the head strap 26 of the goggles 12 may pass. The first and second waterproof housings 14, 16 also may have a first surface 48a, 50a and second surface 48b, 50b, the first surface 48a, 50a being in contact with the user's head and the second surface 48b, 50b being in contact with the head strap 26. The second surface 48b of the first waterproof housing 14 may include the user interface 36.

Continuing to refer to FIG. 1, it is understood that the heart rate measuring apparatus 30 (user interface 36, microcontroller 34, and reflected infrared sensor 32), power source 39, wires 18, and any other necessary components may be housed within a single waterproof housing. The power source 39 is shown in the first waterproof housing 14 in FIG. 1 because it may optionally be included in the first waterproof housing 14, with the second waterproof housing 16 being removed from the biofeedback device 10. All other elements of the biofeedback device 10 are as described for the biofeedback device 10 shown in FIG. 1.

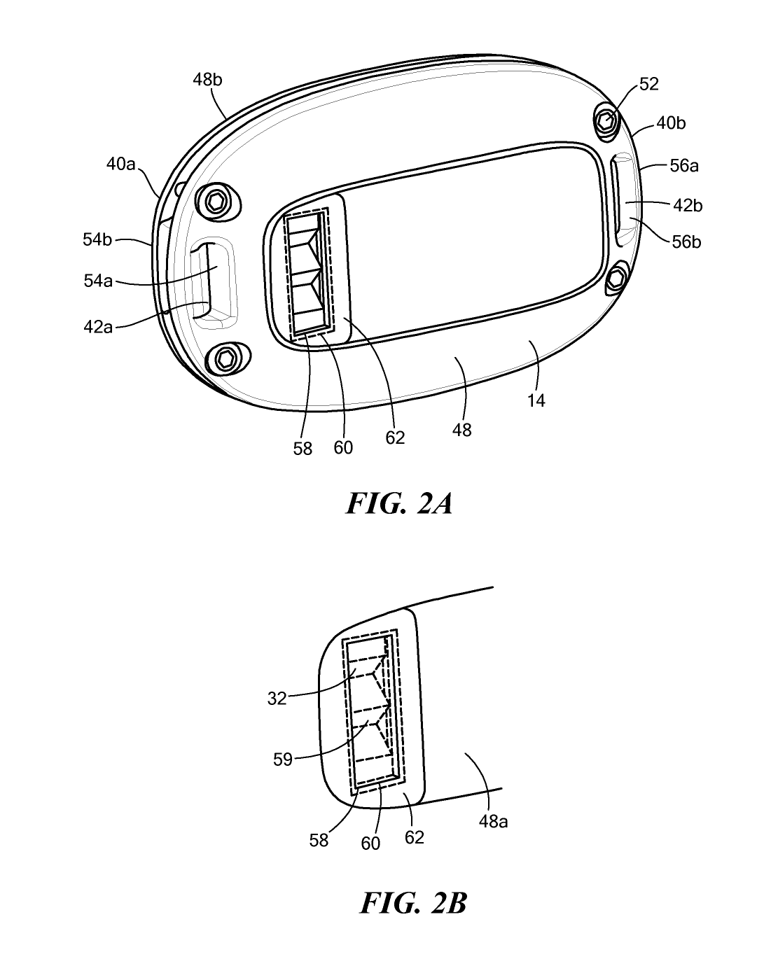

Now referring to FIGS. 2A and 2B, the first surface 48a of the first waterproof housing 14 is shown. One or more screws 52 may be used to seal the housing 14. As is also shown in FIG. 1, the first waterproof housing 14 may have a first end 40a and second end 40b, the first end 40a including a first strap attachment means 42a and the second end 40b including a second strap attachment means 42b. The first and second strap attachment means 42a, 42b each define an opening that may be wide enough to accommodate a typical head strap 26 (for example, the width may be approximately 0.2 cm to 1.0 cm), and may be tall enough to accommodate a typical head strap (for example, the height may be 0.5 cm to 2.0 cm). Each strap attachment means 42a, 42b opening may have an entry 54a, 56a on or adjacent the first surface 48a of the first waterproof housing 14 and an exit 54b, 56b on or adjacent the second surface 48b of the first waterproof housing 14 through which the head strap 26 may pass. For example, to attach the first waterproof housing 14 to the goggles 12 and ensure contact with the user's skin, the head strap 26 may be fed into the entry 54a of the first strap attachment means 42a, then out the exit 54b of the first strap attachment means 42a. The head strap 26 may then be in contact with the second surface 48b of the first waterproof housing 14, passing from the first end 40a to the second end 40b. Finally, the head strap 26 may be fed into the entry 56a and out the exit 56b of the second strap attachment mechanism 42b. The first and second waterproof housings 14, 16 may each be positioned at any location on the strap 26 relative to the user, such as in the back of the user's head or on either side of and immediately adjacent to the eye cups 20a, 20b. Although not shown in FIG. 2A or 2B, it is understood that the second waterproof housing 16, also having a first and second strap attachment means 46a, 46b, may be attached to the goggles 12 in a similar manner.

Continuing to refer to FIGS. 2A and 2B, the first waterproof housing 14 may have a sensor opening 58 through which the reflected infrared sensor 32 is exposed to the skin of the user. The dimensions of the sensor opening 58 may be the same as the dimensions of the area of the sensor 32 that is exposed to the skin. The reflected infrared sensor 32 is entirely disposed within the first waterproof housing 14, whereas the reflected infrared sensor 32 may be substantially coterminous with the sensor opening 58. Because the reflected infrared sensor 32 may be composed of a nonconductive waterproof material, such as Teflon, the sensor opening 58 and at least part of the reflected infrared sensor 32 may be exposed to the water and in direct contact with the skin (as shown in FIG. 2A), or the reflected infrared sensor 32 may be covered by a thin layer 64 of insulation material that allows the transmission of infrared light therethrough, such as silicone 59 (as shown in FIG. 2b). A gasket 60 (such as a typical rubber O-ring) may be included inside the first waterproof housing 14, between the reflected infrared sensor 32 base and the first surface 48a of the first waterproof housing 14, to prevent the entry of water into the housing. Additionally, a portion of the first surface 48a surrounding the outer perimeter of the sensor opening 58 may be covered in a waterproof, opaque material with a relatively high coefficient of friction on skin (approximately 0.3 to 1.0μ), such as rubber. This outer perimeter may help ensure maximum contact and stability between the reflected infrared sensor 32 and the user's skin, thereby increasing the accuracy of the reflected infrared sensor 32's measurements. For simplicity, the area of the first surface 48a of the first waterproof housing 14 is referred to herein as the rubber pad 62, even though it may be composed of a different material.

Referring now to FIG. 3, a second embodiment of the biofeedback device 10 is shown. In this embodiment, the heart rate measuring apparatus 30, power source 39, and one or more wires 18 are entirely disposed within the frame of the goggles 12. The frame of the goggles 12 may be waterproofed like the first and second waterproof housings 14, 16 shown in FIGS. 1, 2A, and 2B and discussed above. The frame of the goggles 12 may include a first arm 64a and a second arm 64b, each arm having a strap attachment means 66 at the terminus. The strap attachment means 66 may comprise a metal or plastic cap and loop through which the head strap 26 may be secured; however, any type of strap attachment means may be used that will securely couple the head strap 26 and goggles 12. The first arm 64a and the second arm 64b each have a first surface 68a, 70a and a second surface 68b, 70b, each first surface 68a, 70a being in contact with the user's head. The heart rate measuring apparatus 30 and the power source 39 may be in electrical communication with each other via one or more wires 18 disposed within a channel defined by the frame of the goggles 12 (if wireless communication is not used). The heart rate measuring apparatus 30 may be entirely disposed within the first arm 64a of the goggles 12, except that the reflected infrared sensor 32 may be exposed to the water or user's skin through an opening 61 on the first surface 68a of the first arm 64a. Similarly, the one or more buttons 37, display screens 38, or other user control features of the user interface 36 are located on the second surface 90b of the first arm 64a, where they are accessible to the user. The power source 39 may be entirely disposed within the second arm 64b of the goggles 12. It is understood, however, that the user interface 36 and heart rate measuring apparatus 30 may be alternatively disposed within the second arm 64b, and the power source 39 may be disposed within the first arm 64a.

Continuing to refer to FIG. 3, the user input may alternatively be located on a remote device 72 in wireless communication with the microcontroller 34 of the heart rate measuring apparatus 30. Thus, the heart rate measuring apparatus 30 in this alternative embodiment may comprise the reflected infrared sensor 32 and microcontroller 34, but not the user interface 36. Including the user interface 36 in a separate from the goggles 12 may allow for a more streamlined design of the biofeedback device 10, as seen in FIG. 4. The remote device 72 may include one or more buttons 37, display screens 38, and other user control elements. The user would enter into the remote device 72 age, weight, target heart rate, workout time, and other data useful in calculating calories burned, workout time, stroke pacing, and other parameters. Additionally, the user interface 36, either disposed within the biofeedback device 10 or remote device 72, could be used for selecting or creating a desired training program. The remote device 72 would wirelessly transmit this data (such as by WiFi, infrared, or Bluetooth® signal) to the microcontroller 34 of the heart rate measuring apparatus 30, which would, in turn, operate the one or more signal elements 28 accordingly (e.g., color of light and/or pace of blinking of LEDs 29). The remote device 72 may include therein a power source 39 that may be rechargeable or single use, for example a small battery such as a hearing aid or watch battery (button cell), and may be waterproof like the first and second waterproof housings 14, 16 shown in FIGS. 1, 2A, and 2B, and discussed above. It should be understood that the remote device configuration may be used with either the integrated or non-integrated heart rate measuring apparatus design (for example, either the biofeedback device 10 of FIG. 1 or the biofeedback device of FIG. 3).

Referring now to FIG. 4, an inside view of the first arm 64a of the goggles 12 is shown. The first surface 68a of the first arm 64a is shown, which includes an opening 61 through which the reflected infrared sensor 32 may be exposed to the user's skin. The reflected infrared sensor 32 may be composed of waterproof materials and therefore may be exposed to the water and in direct contact with the user's skin; however, the reflected infrared sensor 32 may alternatively be covered by a thin layer 64 of insulation material that allows the transmission of infrared light therethrough without distorting the infrared signal (as shown in FIG. 2b).

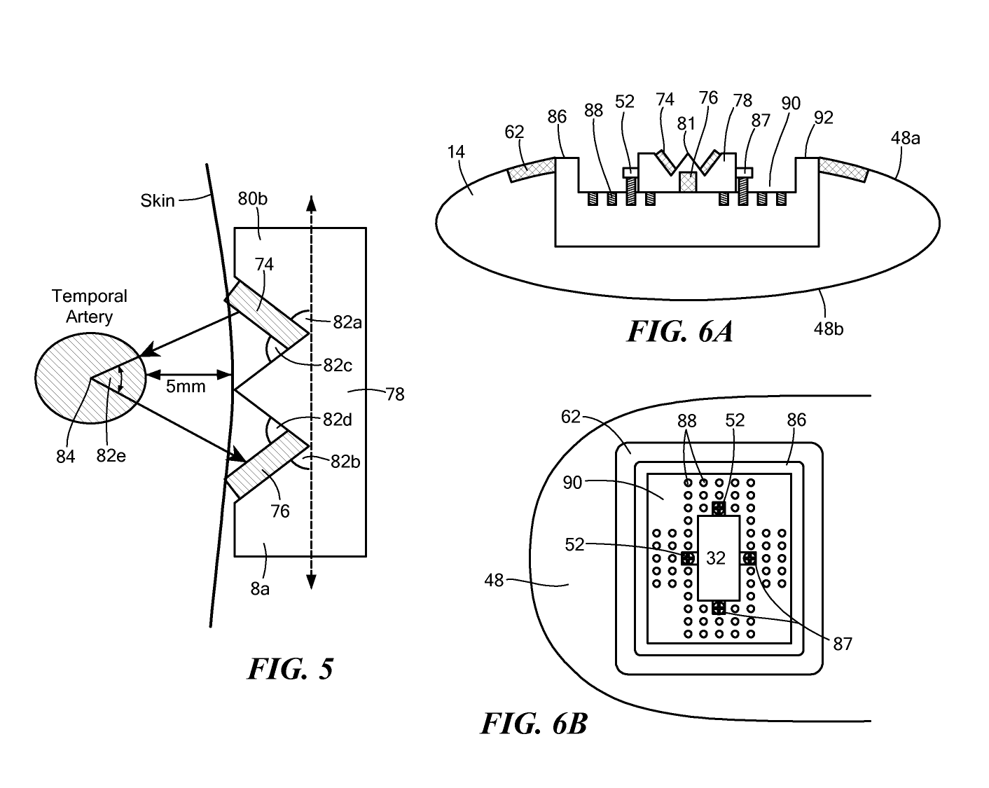

Referring now to FIG. 5, a cross section of the reflected infrared sensor 32 is shown, which may or may not be drawn to scale. The reflected infrared sensor 32 may comprise an infrared emitter 74 (photodiode), an infrared receiver 76 (phototransistor), and sensor base 78 having a first end 80a and a second end 80b, the sensor base 78 defining a shield element 81 to prevent the possible interference between the emitted and received infrared signals (i.e. to prevent the infrared light emitted from the infrared emitter 74 from directly entering the infrared receiver 76 without first being reflected from the target reflection point 84). The shield element 81 may be any size and shape sufficient to prevent the infrared signal interference, such as triangular shape. The reflected infrared sensor 32 may be composed of a nonconductive material, such as Teflon, to prevent interference with the current in the infrared emitter 74 and infrared receiver 76. Additionally, the material may be opaque and non-reflective in order to block any light that can interfere with the infrared light emitted by the infrared emitter 74 and/or distort the signal received by the infrared receiver 76. For simplicity, the term “reflected infrared sensor” used herein includes the infrared emitter 74, infrared receiver 76, and shield element 81. The reflected infrared sensor 32 may be placed in contact with the user's skin; the temporal artery is located approximately 5 mm beneath the skin of the temple.

Continuing to refer to FIG. 5, the cross-sectional view of the reflected infrared sensor 32 may resemble the letter “W.” The infrared emitter 74 may be positioned at a first angle 82a measured in relation to an axis running from the first end 80a of the sensor base 78 to the second end 80b of the sensor base 78, and the infrared receiver 76 may be positioned at a second angle 82b measured in relation to said axis. Further, the infrared emitter 74 and the shield element 81 may define a third angle 82c, and the shield element 81 and the infrared receiver 76 may define a fourth angle 82d. The reflected infrared sensor 32 configuration may be determined for any target reflection point 84. For example, the angle between the infrared emitter 74 and the shield element 81 may be set at 45 degrees. Next, a point 5 mm from the outer edge of the infrared emitter 74 may be used as the reflection point because the temporal artery is located an average of 5 mm beneath the skin of the temple (as shown in FIG. 5). Then, the distance between the infrared emitter 74 and infrared receiver 76 may be adjusted until an oscilloscope measurement of the infrared signal is of the highest amplitude, which means the location of the infrared receiver 76 would ensure optimal receipt of the infrared light. The degree of emission (the fifth angle 82e) of the infrared light from the infrared emitter 74 may also be determined, based on the relative positions of the infrared emitter 74, infrared receiver 76, and the target reflection point 84.

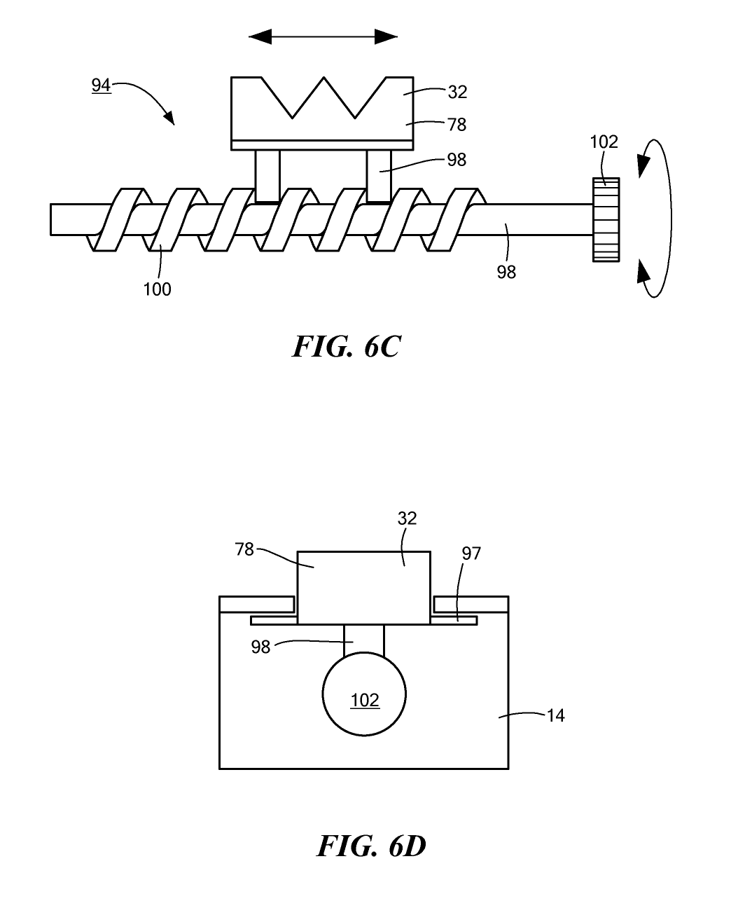

Referring now to FIGS. 6A, 6B, and 6C, the reflected infrared sensor 32 may be adjusted by the user horizontally (along an x-axis), vertically (along a y-axis), or a combination of horizontally and vertically to a distance of, for example, 1 cm. Since there are minimal variations between the location of the temporal artery between one person and another, the reflected infrared sensor 32 may be mounted within the waterproof housing (either in, for example, the first waterproof housing 14 or the first arm 64a of the goggles 12) in such a way that allows for the positioning of the reflected infrared sensor 32 by tightening or loosening one or more screws 52, while still preventing the entry of water into the waterproof housing. If the reflected infrared sensor 32 does not detect the user's heart rate, the one or more signal elements 28 will not broadcast a visual, auditory, or tactile heart rate signal to the user, but may instead emit a blinking red light. In this case, the user may adjust the reflected infrared sensor 32 until heart rate is detected. Unlike other heart rate measuring devices, the reflected infrared sensor 32 may not be easily repositioned by repositioning the entire device 10, because the goggles 12 must be fitted over the eyes of the user and thus may not be able to accommodate movement of a fixed sensor. Exemplary methods of adjusting the reflected infrared sensor are shown in FIGS. 6A, 6B, and 6C.

FIG. 6A shows a cross-sectional view of the first waterproof housing 14 with a panel-type sensor adjustment mechanism 86. The reflected infrared sensor 32, or a plurality of reflected infrared sensors 32, may be coupled to the panel-type sensor adjustment mechanism 86 by one or more screws 52 that may be screwed into any of a plurality of screw holes 88 located on the surface 90 of the panel-type sensor adjustment mechanism 86. The screw holes 88 may terminate at least partially through, but do not continue all the way through, the panel-type sensor adjustment mechanism 86, which prevents water from entering the first waterproof housing 14. The panel-type sensor adjustment mechanism 86 may be coupled to the first waterproof housing 14 such that only the outer rim 92 of the panel-type sensor adjustment mechanism 86 may be flush with the first surface 48a of the first waterproof housing 14, with the surface 90 of the panel-type sensor adjustment mechanism 86 being recessed. Similarly, the portion of the reflected infrared sensor 32 that is in contact with the skin may be substantially coplanar with the first surface 48a of the first waterproof housing 14.

Referring now to FIG. 6B, the panel-type sensor adjustment mechanism 86 may be adjusted horizontally (along an x-axis), vertically (along a y-axis), or a combination of horizontally and vertically by unscrewing the one or more screws 52 from any of a plurality of screw holes 88, moving the reflected infrared sensor 32 along the surface 90 of the panel-type sensor adjustment mechanism 86, and replacing the one or more screws 52 into the corresponding one or more screw holes 88. The sensor base 78 may also have one or more flanges 87 having one or more screw holes 88 that align with the one or more screw holes 88 on the surface 90 of the panel-type sensor adjustment mechanism 86. The entire surface 90 and outer rim 92 of the panel-type sensor adjustment mechanism 86 are waterproof and may be exposed to water.

Alternative or additional to the method of adjusting the reflected infrared sensor 32 shown in FIGS. 6A and 6B, a spiral-type sensor adjustment mechanism 94 may be included. In the spiral-type sensor adjustment mechanism 94, reflected infrared sensor 32 may or may not be coupled to a surface having a plurality of screw holes 88. Instead, the infrared sensor 32 may be coupled to an adjustment plate 97 disposed within the first waterproof housing 14. The sensor base 78 may include one or more feet 98 that may be in contact with a shaft 98 having a spiraled threading 100 (for example, a screw). The one or more feet 96, the shaft 98, and the spiraled threading 100 may be entirely disposed within the first waterproof housing 14. Coupled to one end of the shaft 98 may be a knob 102, which is not disposed within the first waterproof housing 14, but is instead accessible to the user. When the user turns the knob either clockwise or counterclockwise, the spiraled threading 100 engages the feet 96 to move the reflected infrared sensor 32 along either the x-axis or the y-axis (for example, to a distance of 1 cm from the centerpoint in either direction), depending on the axis on which the spiral-type sensor adjustment mechanism 94 is disposed. It is understood that the sensor adjustment mechanisms 86, 94 of FIGS. 6A-6C could be similarly disposed within other waterproof housings, for example, the first arm 64a of the goggles 12.

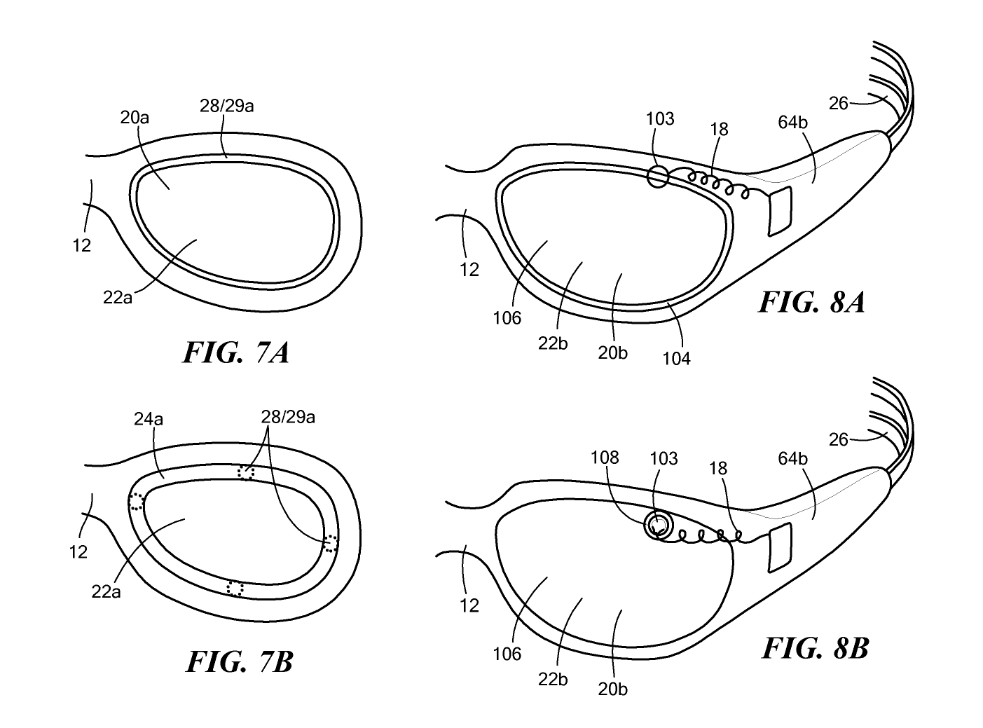

Referring now to FIGS. 7A and 7B, the one or more signal elements 28 are shown. FIG. 6A shows a continuous rope of clear tubing with multiple LEDs 29 therein 29a, and FIG. 7B shows discrete LEDs 29b. The clear tubing may contain one or more LEDs 29, and is referred to herein as a “rope-type LED light” 29a. Each eye cup 20a, 20b includes a lens 22a, 22b, which is the surface of the eye cup that is disposed directly in front of the user's eye. The rope-type LED light 29a may be at least partially disposed about the inner circumference of at least one of the first and second eye cups 20a, 20b either adjacent to or on the lens 22a, 22b. Included in the first eye cup 20a is a first lens 22a, and included in the second eye cup 20b is a second lens 22b.

The rope-type LED light 29a may be entirely disposed about a circumference of at least one of the first and second lenses 22a, 22b. For example, FIG. 7A shows the rope-type LED light 29a disposed about the entire inner circumference of the first eye cup 20a. Alternatively, the rope-type LED light 29a may be disposed within or underneath at least one of the first and second eye cup gaskets 24a, 24b, at least partially disposed about the inner circumference of the eye cup 20a, 20b where the eye cup 20a, 20b is coupled to the eye cup gasket 24a, 24b. Depending on the placement of the rope-type LED light 29a, the user may either perceive a direct light or an indirect light. When the rope-type LED light 29a is disposed within at least one of the first and second eye cup gaskets 24a, 24b, the light may be a diffuse light that is reflected from the inside of the eye cup 20a, 20b and may give the effect of illuminating the entire eye cup with color. No matter what the placement of the rope-type LED light 29a, the user should be able to perceive the color and/or blinking of the light without undue effort.

Continuing to refer to FIG. 7B, one or more discrete LEDs 29b are shown. The discrete LEDs 29b may be located at any position about the inner circumference of at least one of the first and second eye cups 20a, 20b, either adjacent to or on the first and/or second lens 22a, 22b. Any number of discrete LEDs 29b may be used. The discrete LEDs 29b may be equidistant from one another, or they may be grouped together at any point in the first and/or eye cup 20a, 20b. Alternatively, the discrete LEDs 29b may be disposed within or underneath at least one of the first and second eye cup gaskets 24a, 24b (as shown in FIG. 7B). Depending on the placement of the discrete LEDs 29b, the user may either perceive a direct light or an indirect light. When the discrete LEDs 29b are disposed within at least one of the first and second eye cup gaskets 24a, 24b, the light may be a diffuse light that is reflected from the inside of the eye cup 20a, 20b and may give the effect of illuminating the entire eye cup with color. No matter what the placement of the discrete LEDs 29b, the user should be able to perceive the color and/or blinking of the light without undue effort.

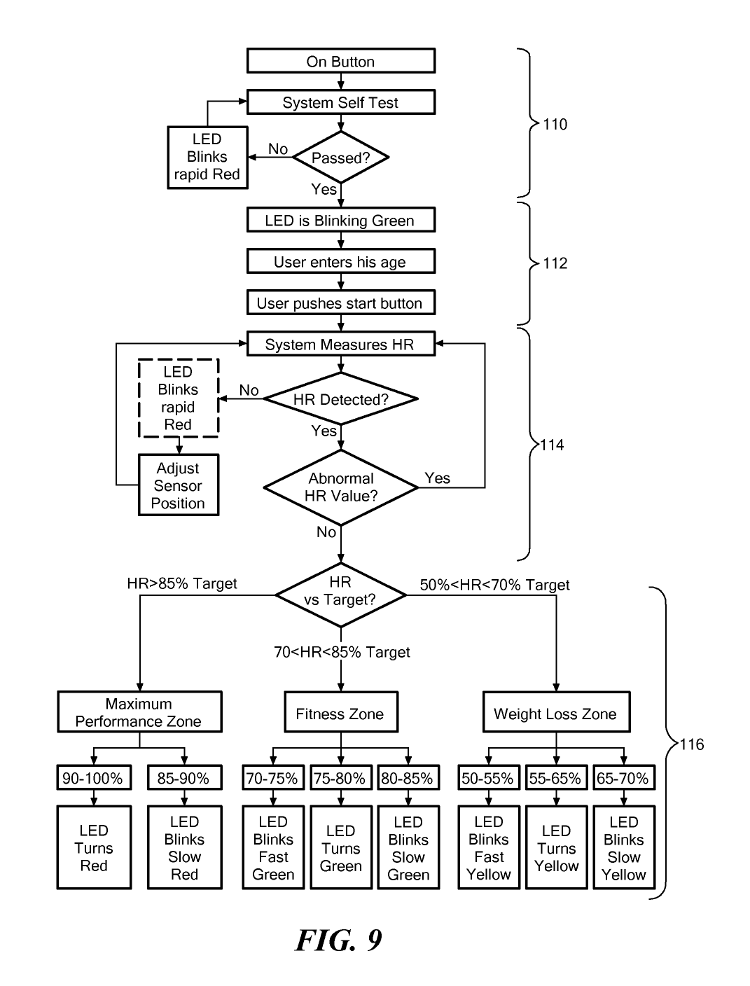

Referring now to FIGS. 8A and 8B, the one or more signal elements 28 may alternatively be coupled to or housed in a positionable element 103 that the user may place in any desired position on the biofeedback device 10. Such a housing may have such attachment means as a clip, adhesive junction, suction cup, malleable arm coupled to the goggles, or any other suitable means. For example, FIG. 8A shows the goggles 12 having an eye cup track 104 that may be disposed at least partially about the circumference of the outer surface 106 of one or both eye cups 20a, 20b. The one or more signal elements 28 may be removably coupled to the eye cup track 104, such as by a clip. FIG. 9B shows the one or more signal elements 28 coupled to a suction cup 108 that may be removably attached to the outer surface 106 of one or both eye cups 20a, 20b. Regardless of the type of positionable element 103 used, the positionable element 103 may be in electrical communication with the power source 39 and microcontroller 34 via one or more flexible wires 18 that may be at least partially disposed on the outside of the goggles 12 (not within a waterproof housing).

Referring now to FIG. 9, an exemplary communication scheme of the one or more signal elements 28 is shown. In FIG. 9, a visual signal element is contemplated, specifically, an LED display. Three colors of LEDs 29 may be used to represent the three training zones (weight loss, fitness, and maximum performance). It is understood that more colors may be used, depending on the number of training zones to be represented. Additionally, the LEDs 29 may emit a steady light only, or may emit a steady light or a blinking light to represent upper and lower ends of the represented training zones. The LEDs 29 may emit a blinking red light if the reflected infrared sensor 32 does not detect a heart rate. The presence of a blinking light will communicate to the user that the unit has sufficient power, but that the sensor is not in the optimal location for detecting heart rate. Further, the color of the light and its status (blinking or steady) easily communicate heart rate to the user without requiring the user to read small numbers or pause swimming to look at a watch or similar device.

FIG. 9 shows an example of this system: after a boot up sequence 110, the user may enter data into the user interface 36 (such as age, weight, or desired workout program), the process referred to as “user data entry” 112. The heart rate measuring apparatus 30 may then detect and measure the user's heart rate, and the user may manually adjust the position of the reflected infrared sensor 32 if no heart rate is detected. This process is referred to as “heart rate detection and adjustment” 114. After heart rate detection and measurement 114, heart rate measuring apparatus 30 may then compare the user's heart rate to the user's target heart rate and communicate the result to the one or more signal elements 28, a processed referred to as “comparison and display” 116.

FIG. 9 also shows an exemplary comparison and display 116 process, in which the weight loss zone is typically a heart rate of 50% to 70% of the maximum heart rate, and may be represented by one or more green LEDs 29. The green LEDs 29 may blink slowly in the 50% to 55% range (lower end of the zone), may glow steadily in the 55% to 65% range (middle of the zone), and may blink quickly in the 65% to 75% range (upper end of the zone). The fitness zone is typically a heart rate of 70% to 85% of the maximum heart rate, and may be represented by one or more yellow LEDs 29. The yellow LEDs 29 may blink slowly in the 70% to 75% range (lower end of the zone), may glow steadily in the 75% to 80% range (middle of the zone), and may blink quickly in the 80% to 85% range (upper end of the zone). The maximum performance zone is typically a heart rate of 85% of the maximum heart rate and above, and may be represented by one or more red LEDs 29. The red LEDs 29 may glow steadily in the 85% to 90% range (lower end of the zone), and may blink slowly at heart rates above 90% of the maximum heart rate (middle and upper end of the zone). Depending on the LEDs 29 used, any number of color options may be available for a single LED bulb (such as when multi-color LEDs 29 are used, or when the signal display element comprises multiple LEDs 29 of various colors). The user interface 36 may include a means by which the user may adjust the LED display correlated to heart rate. For example, the user may prefer blue LEDs 29 for the weight loss zone, red LEDs 29 for the fitness zone, and green LEDs 29 for the maximum performance zone. Additionally, the user may also use the user interface 36 to specify a steady LED glow without blinking, or may desire to set the speed of the blinking to match a target swim stroke pace.

It should be understood that the microcontroller 34 may measure and record other types of biofeedback data in addition to heart rate, and may also be able to measure non-biofeedback data. For example, the microcontroller 34 of the biofeedback device 10 may additionally comprise circuitry for performing the functions of a chronometer, timer, lap counter, distance measurement device, calorie counter, blood oximeter, and wireless transmitter (such as a Bluetooth® device).

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed:

1. A biofeedback device comprising
a heart rate measuring apparatus comprising a reflected infrared sensor, the sensor including a sensor base having a first end and a second end, an infrared light emitter and an infrared light receiver, the infrared light emitter being coupled to the sensor base at a first selected angle in relation to an axis running from the first end to the second end of the sensor base, and the infrared light receiver being coupled to the sensor base at a second selected angle in relation to said axis, and the infrared light emitter and the infrared light receiver being spaced apart from each other at a selected distance determined by the target reflection point; and
a power source in electrical communication with the heart rate measuring apparatus.

2. The biofeedback device of claim 1, wherein
the infrared light emitter emits infrared light an at intensity sufficient to penetrate human skin to image blood vessels; and
the sensor base defines a triangular-shaped shield element that prevents emitted infrared light from traveling directly into the infrared light receiver, the shield element being disposed between the infrared light emitter and the infrared light receiver.

3. The biofeedback device of claim 2, wherein the heart rate measuring apparatus further comprises
a microcontroller in electrical communication with the reflected infrared sensor and the power source, the microcontroller including one or more filters and one or more amplifiers;
a the user interface in electrical communication with the microcontroller; and
one or more signal elements in electrical communication with the microcontroller.

4. The biofeedback device of claim 3, wherein the biofeedback device further comprises a pair of swim goggles comprising a first and second eye cup.

5. The biofeedback device of claim 4, wherein the reflected infrared sensor outputs a signal to the microprocessor that filters, amplifies, performs calculations on the received signal and outputs a signal to the one or more signal elements, and the one or more signal elements receive the output signal from the microprocessor and are actuated in response thereto, and wherein the one or more signal elements are selected from the group consisting of
visual signal elements;
auditory signal elements; and
tactile signal elements.

6. The biofeedback device of claim 5, wherein the one or more signal elements are visual signal elements and include one or more light-emitting diodes that cast light in the user's field of vision, wherein the light-emitting diodes have a configuration selected from the group consisting of
discrete light-emitting diodes; and
light-emitting diodes disposed within a transparent tube positioned about a circumferential portion of at least one of the first and second eye cup of the swim goggles.

7. The biofeedback device of claim 3, wherein the reflected infrared sensor, microcontroller, and user interface are disposed within one or more waterproof housing units.

8. The biofeedback device of claim 3, wherein the biofeedback device further includes a remote device and wherein both the microcontroller and remote device include a wireless interface and are in wireless communication with each other, the reflected infrared sensor and microcontroller being disposed within a waterproof housing unit and the user interface being disposed within the remote device.

9. The biofeedback device of claim 6, wherein the intensity and duration of the light emitted by the light-emitting diodes is controllable through the user interface.

10. The biofeedback device of claim 6, wherein the light-emitting diodes emit one of three distinct colors, wherein a first distinct color is emitted when the user's heart rate is within 50% to 70% of the user's maximum heart rate, a second distinct color is emitted when the user's heart rate is within 70% to 85% of the user's maximum heart rate, and a third distinct color is emitted when the user's heart rate is 85% or greater of the user's maximum heart rate.

11. The biofeedback device of claim 10, wherein the light-emitting diodes
emits a first distinct color and blinks slowly when the user's heart rate is within approximately 50% to 55% of the user's maximum heart rate;
steadily emits a first distinct color when the user's heart rate is within approximately 55% to 65% of the user's maximum heart rate;
emits a first distinct color and blinks rapidly when the user's heart rate is within approximately 65% to 70% of the user's maximum heart rate;
emits a second distinct color and blinks slowly when the user's heart rate is within approximately 70% to 75% of the user's maximum heart rate;
steadily emits a second distinct color when the user's heart rate is within approximately 75% to 80% of the user's maximum heart rate;
emits a second distinct color and blinks rapidly when the user's heart rate is within approximately 80% to 85% of the user's maximum heart rate;
steadily emits a third distinct color when the user's heart rate is within approximately 85% to 90% of the user's maximum heart rate; and
emits a third distinct color and blinks slowly when the user's heart rate is above approximately 90% of the user's maximum heart rate.

12. A reflected infrared sensor comprising
a sensor base having a first end and a second end;
an infrared light emitter coupled to the sensor base at a first selected angle in relation to an axis running from the first end to the second end of the sensor base;
an infrared light receiver coupled to the sensor base at a second selected angle in relation to the axis running from the first end to the second end of the sensor base; and
a light-blocking element disposed between the emitter and receiver, defining a third selected angle between the infrared light emitter and the light-blocking element and defining a fourth selected angle between the light-blocking element and the infrared light receiver; and
an infrared sensor adjustment mechanism,
wherein the infrared light emitter emits a beam of infrared light at a fifth selected angle in relation to the surface of the infrared light emitter, the emitted beam of infrared light being of an intensity sufficient to penetrate human skin to a target reflection point located at a selected depth beneath the skin.

13. The reflected infrared sensor of claim 12, wherein the reflected infrared sensor is disposed within a waterproof housing unit that further includes a microcontroller and a power source disposed therein, the microcontroller and power source being in electrical communication with the reflected infrared sensor, and the microcontroller including one or more filters that filter electromagnetic interference and ambient light from the reflected infrared sensor signal, and one or more amplifiers that amplify the filtered signal.

14. The reflected infrared sensor of claim 13, wherein the microcontroller further includes a wireless interface, and wherein the waterproof housing unit has a configuration selected from the group consisting of
the waterproof housing unit is removably coupled to a pair of swim goggles having a first and second eye cup, and further includes a user interface in electrical communication with the microcontroller and power source;
the waterproof housing unit is removably coupled to a pair of swim goggles having a first and second eye cup, the microcontroller being in wireless communication with a remote device including a user interface;
the waterproof housing unit further includes a user interface in electrical communication with the microcontroller and power source, the waterproof housing unit consisting of a pair of swim goggles having a first and second eye cup; and
the waterproof housing unit consists of a pair of swim goggles having a first and second eye cup, the microcontroller being in wireless communication with a remove device including a user interface.

15. The reflected infrared sensor of claim 14, wherein the microcontroller is further in electrical or wireless communication with one or more signal elements.

16. The reflected infrared sensor of claim 15 wherein one or more signal elements comprise one or more light-emitting diodes have a configuration selected from the group consisting of
discrete light-emitting diodes disposed within or adjacent to the eye cup;
light-emitting diodes disposed within a transparent tube positioned about a circumferential portion of at least one of the first and second eye cup; and
discrete light-emitting diodes coupled to a positionable element.

17. The reflected infrared sensor of claim 16, wherein the light-emitting diodes emit one of three or more distinct colors, wherein a first distinct color is emitted when the user's heart rate is within 50% to 70% of the user's maximum heart rate, a second distinct color is emitted when the user's heart rate is within 70% to 85% of the user's maximum heart rate, and a third distinct color is emitted when the user's heart rate is 85% or greater of the user's maximum heart rate.

18. The reflected infrared sensor of claim 17, wherein the positionable element is selected from the group consisting of
a positionable element that is removably coupled to an eye cup track that is at least partially disposed about the outer surface of one or both eye cups; and
a suction cup.

19. The reflected infrared sensor of claim 17, wherein the light-emitting diode
emits a first distinct color and blinks slowly when the user's heart rate is within approximately 50% to 55% of the user's maximum heart rate;
steadily emits a first distinct color when the user's heart rate is within approximately 55% to 65% of the user's maximum heart rate;
emits a first distinct color and blinks rapidly when the user's heart rate is within approximately 65% to 70% of the user's maximum heart rate;
emits a second distinct color and blinks slowly when the user's heart rate is within approximately 70% to 75% of the user's maximum heart rate;
steadily emits a second distinct color when the user's heart rate is within approximately 75% to 80% of the user's maximum heart rate;
emits a second distinct color and blinks rapidly when the user's heart rate is within approximately 80% to 85% of the user's maximum heart rate;
steadily emits a third distinct color when the user's heart rate is within approximately 85% to 90% of the user's maximum heart rate;
emits a third distinct color and blinks slowly when the user's heart rate is above approximately 90% of the user's maximum heart rate; and
emits a third distinct color and blinks rapidly when the sensor does not detect the user's heart rate.

20. The reflected infrared sensor of claim 16, wherein the color, intensity, and duration of light emitted by the one or more light-emitting diodes is controllable through the user interface.

21. The reflected infrared sensor of claim 13, wherein the microcontroller further includes circuitry capable of keeping time, measuring time elapsed, measuring distance traveled, counting calories burned, and measuring blood oxygen levels.

22. The reflected infrared sensor of claim 13, wherein the infrared sensor adjustment mechanism is selected from the group consisting of
a panel-type sensor adjustment mechanism comprising a surface having a plurality of screw holes that align with one or more screw holes in the sensor base, wherein the reflected infrared sensor is coupled to the surface of the sensor adjustment mechanism with one or more screws that engage the screw holes of both the sensor base and the surface of the sensor adjustment mechanism;
a spiral-type sensor adjustment mechanism including a shaft with threading disposed within the waterproof housing and a knob disposed outside of the waterproof housing, wherein the sensor base includes feet that are engageable by the threading; and
a combination of both panel-type and spiral-type sensor adjustment mechanisms.
